(19) United States
(12) Patent Application Publication
(10) Pub. No.: US 2009/0216219 A1
(43) Pub. Date: Aug. 27, 2009

(54) PHOTON THERAPY DEVICE

(75) Inventors: Cornelia Maria Venter, Pretoria (ZA); Cornelius Johannes Du Plooy, Pretoria (ZA); Dragomir Krdzalic, Pretoria (ZA)

Correspondence Address: SMITH HOPEN, PA, 180 PINE AVENUE NORTH, OLDSMAR, FL, 34677, US

(73) Assignee: Photon Therapy Systems (Pty) Ltd., Pretoria (ZA)

(21) Appl. No.: 11/914,842

(22) PCT Filed: May 17, 2006
(86) PCT No.: PCT/ZA06/00070

§ 371 Date: Nov. 19, 2007

(30) Foreign Application Priority Data
May 17, 2005 (ZA) 2005/02343

(51) Int. Cl. A61B 18/20; A61N 5/06

(52) U.S. Cl. 606/11; 607/90

(57) ABSTRACT

The invention provides a controller unit for controlling the emission of at least on photon emitted source, the controller unit includes a central processing unit pre-programmed with selectable pre-programmed photonic emission protocols. The photonic emission protocols include parameters for regulating photonic emission from photon emitting sources, with the protocols having a pulsed emission mode for about 30% of the duration of each protocol, and a continuous emission mode for about 70% of the duration of each protocol, with a pulse rate selected from a range of frequencies of between 120 Hz and 20,000 Hz when in the pulsed emission mode.

### CROSS REFERENCE

This application is a 371 National Phase Entry of PCT/ZA2006/000070 file May 17, 2006 which claims priority to South African Patent Application No. ZA 2005/02343, filed May 17, 2005.

THIS INVENTION relates to a photon therapy device. More particularly, this invention relates to a controller unit for a photon therapy device for use in photobiomodulation therapy, and to the use of a photon therapy device in the treatment of medical conditions.

### BACKGROUND OF THE INVENTION

Photobiomodulation, also known as photon therapy, is becoming an increasingly common technique in treating a variety of ailments. Typically, photon therapy is administered using light or photon therapy devices, which may be grouped according to the type of light produced into two distinct groups, viz. low level laser and LED. Usually, such photon therapy units have a variety of individually adjustable parameters which may be adjusted and optimized by a skilled or trained clinician for treating musculoskeletal or dermatological disorders. These devices are usually limited to clinical practice and are not suitable for use by less sophisticated users. In addition, there is a need for such devices in rural areas where there is no readily available source of electricity and where the users may be relatively unsophisticated.

### SUMMARY OF THE INVENTION

According to one aspect of the invention, there is provided a controller unit for controlling the emission of at least one photon emitting source in communication with the controller unit, the controller unit including: at least one central processing unit pre-programmed with at least one selectable photonic emission protocol, said photonic emission protocol having included therein parameters for regulating photonic emission from the at least one photon emitting source, wherein the at least one pre-programmed emission protocol has a pulsed emission mode and a continuous emission mode, with a pulse rate elected from a range of frequencies of between 120 Hz and 20,000 Hz when in the pulsed emission mode; and selection means for allowing a user to select the at least one pre-programmed photonic emission protocol.

Accordingly, the invention extends to a photon therapy device, the photon therapy device including the controller unit of the invention and at least one photon emitting source.

The at least one pre-programmed photonic emission protocol may include pulsed photonic emission for a time period equivalent to about 15 to 50% of the total treatment time for the desired photon emission protocol, preferably about 20 to 40%, most preferably about 30% of the desired photon emission protocol time.

The parameters included in the at least one pre-programmed photonic emission protocol may include the pulse frequency, dose, intensity, irradiation time, and continuous or pulsed emission mode of the at least one photon emitting source.

The at least one photon emitting source may be a light emitting diode (LED), an LED array, or a plurality of LED'S. The LED may be a high power LED array. More specifically, the photon emitting source may be a commercially available 20 V LED.

The controller unit may include circuitry for powering a second photon emitting source in synchronization with the first photon emitting source. Alternatively, or additionally, the controller unit may include circuitry for powering the second photon emitting source separately from the first photon emitting source.

The wavelengths of light produced by the photon emitting sources may be near coherent and may be between about 500 nm and about 900 nm, preferably between about 400 and 700 nm, most preferably between about 620 and 660 nm. In one embodiment of the invention, the wavelength may be in the order of about 640 nm (210%).

Power output of the photon emitting sources may be about 50 to 150 mW in continuous wave mode, preferably about 80 to 120 mW, most preferably about 100 mW. Power output of the photon emitting sources may be about 30 to 70 mW in pulsed mode, preferably about 40 to 60 mW, most preferably about 50 mW.

The second photon emitting source may have a wavelength or power output differing from 15 that of the first photon emitting source.

In a preferred embodiment of the invention, the controller unit may include a plurality of pre-programmed protocols, each being selectable by way of a separate button associated with each protocol.

The photon emitting sources may be activated by a user selecting the desired protocol by selecting a button for the desired protocol on the control unit and pushing the button an appropriate number of times to activate either the first photon emitting source, the second photon emitting source, or both photon emitting sources simultaneously.

Activation of the respective photon emitting sources may be signalled to the user by the selected button or an associated marker flashing at a pre-determined flashing rate, or until a start or stop command has been selected. The controller unit includes additional circuitry to detect whether the second photon emitting source has been connected to the controller unit, thereby allowing the additional circuitry to be activated only once the second photon source has been connected to the controller unit.

According to a still further aspect of the invention, there is provided use of a photon therapy device of the invention in accelerating healing of a wound, lesion or medical condition.

The lesion or condition may be selected from the group consisting of, but not limited to: venous ulcers; mouth ulcers; cuts; abrasions; scratches; blisters; surgical wounds; burns; bedsores; superficial bruising; lip wounds; mosquito and other insect bites; contact dermatitis; dermatitis after cancer radiation treatment; accelerating of healing of skin grafts; hemorrhoids (protruding); Burger's disease; Raynaud's disease; neuropathy; 10 herpes simplex infections; herpes zoster infections; cold sores; shingles; post-herpetic neuralgia; Bell's palsy; warts; chicken pox; eczema; boils; tooth abscesses; wounds following tooth extraction; pain following root canal procedures; hypersensitive gums; gingivitis; hypersensitive dentine; post-operative pain following dental implantation; oral mucositis; upper respiratory tract infections and swelling; tonsillitis; and acne, acne-related 15 lesions or other seborrhoeic skin conditions, seromas, skin hematomas, fine lines and wrinkles, crow's feet, blemishes, sun damaged skin, brown age spots, irregular pigmentation, and skin coarseness, scleroderma, sunburn, liver spots, alopecia, cellulites, general swelling (including post-operative swelling), lymph edema, lymphatic damage, mastitis, post traumatic swelling, inflammation, swelling in the ear canal, neuropathy, 20 burning of hands and feet, carpal tunnel syndrome, peripheral nerve injuries, sciatica, vascular pathology, varicose veins, vasculitis, thrombophlebitis, muscle trauma, muscle spasms (e.g. neck and back spasms), fibromyalgia, repetitive/over-use syndromes, muscle strains, muscle contusions, muscle surgery, and muscle pain/myalgia, ligament, tendon and fascia injuries such as strains, sprains, inflammation, bruising, post-operative 25 orthopedic conditions, ITB/runner's knee, fasciitis, plantar fasciitis, tendonitis, synovitis, tension headache and migraine resulting from muscle spasms, joint injuries, inflammation, athralgia (painful joints), tennis and golfer's elbow, shin splints, trigger points, rotator cuff syndromes, acupuncture points, trigger points, fibrositis, arthritic conditions e.g. DJD (degenerative joint disease), rheumatoid arthritis, gout, cartilage damage (e.g. 30 meniscus bruising/tears), disc injuries/degeneration, synovitis, metatarsal pain and inflammation, calcaneal spur, bunions, hammer-and-claw toe, fractures, stress fractures, tempero-mandibular conditions, fibro-athralgia, spinal column pain associated with pain in the neck and back, oral and mucosal pathologies such as skin breaks following dental work, acute and chronic sinusitis, rhinitis/rhinopathy, inflammatory skin conditions, gingivitis, hypersensitive dentine, oral mucositis, oral wounds, lip wounds, nose fractures, post-operative ear, nose, throat and oral surgery, mouth ulcers, tooth abscesses, pain following root canal procedures, throat infections, painful mouth nerve conditions, conditions that involve an infective process, e.g. certain bacterial, viral, and fungal 5 infections, cold sores (on lips, genitalia or inside mouth), shingles, post-herpetic neuralgia, tooth abscesses, cysts (inflamed or seborrhoiec), warts, osteoitis/bone infection, peri-anal abscesses, genitalia infections, and vaginitis, swimmers/tropical ear, tinnitus, athlete's foot, skin and fungal infections, veterinary applications such as othematoma, hot spots, pododermatitis, stomatitis, hygromas, rodent ulcers, useful in effecting skin re-pigmentation, over use syndromes in equines, acral lick granuloma, bite wounds, cysts, hyaloma tick bite wounds, keratosis, and de-gloving wounds.

Depending on the desired pre-programmed emission protocol, the predetermined photonic emission parameters may provide a dose in a range having a lower value of about 0.5 J/cm2 tissue and a higher value of about 20 J/cm2 tissue.

Typically, when treating an acute (0-21 days old) wound the dose may be in a range having a lower value of about 0.5 J/cm2 tissue, more preferably about 1 J/cm2. The range may have an upper value of 4 J/cm2 tissue, more preferably about 3 J/cm2 tissue. Most preferably, the dose may be about 2 J/cm2 tissue.

This treatment protocol may also be suitable for treating: Cuts; scratches; scrapes; blisters; surgical wounds; burns; bedsores; superficial bruising; lip wounds; mosquito and other insect bites; contact dermatitis; dermatitis following cancer radiation therapy; and to accelerate healing of skin grafts, swelling, wrinkles, mucositis, pain, keloids, post-dental procedures, sunburn skin, skin degration, de-gloving wounds, and is useful in effecting skin re-pigmentation.

Typically, when treating a chronic (>21 days old) wound, the dose may be in a range having a lower value of about 1 J/cm2 tissue, more preferably about 2 J/cm2. The range may have an upper value of 6 J/cm2 tissue, more preferably about 4 J/cm2 tissue. Most preferably, the dose may be about 4 J/cm2 tissue.

This treatment protocol may also be suitable for treating the following conditions: haemorrhoids (protruding); Burger's disease; Raynaud's disease; neuropathy; ulcers (diabetic and venous), infected wounds, superficial bruises, bed soreslpressure wounds, haemorrhoids (protruding), eczema, psoriasis, blemishes, skin coarseness, scleroderma, sunburnt skin.

Typically, when treating oral pathologies such as mucositis, mouth ulcers, or the like, the dose may be in a range having a lower value of about 1 J/cm2 tissue, more preferably about 2 J/cm2. The range may have an upper value of 5 J/cm2 tissue, more preferably about 4 J/cm2 tissue. Most preferably, the dose may be about 3 J/cm2 tissue.

This treatment protocol may also be suitable for treating: tooth abscesses; wounds following tooth extraction; pain following root canal procedures; hypersensitive gums; gingivitis; hypersensitive dentine; post-operative pain following dental implantation; oral 15 mucositis; upper respiratory tract infections and swelling; and tonsillitis, dental implants, or skin breaks following dental work, acute and chronic sinusitis, rhinitislrhinopathy, inflammatory skin conditions, gingivitis, hypersensitive dentine, oral mucositis, and oral wounds, lip wounds, nose fractures, post operative ear, nose, throat and oral surgery, mouth ulcers, tooth abscesses, wounds after tooth extraction, pain after root canals, hypersensitive gums, gingivitis, hypersensitive dentine, post-operative pain after dental implants, mucositis after radiation, sore throat and tonsils and painful mouth nerve conditions.

Typically, when treating infections, abscesses, or acne, the dose may be in a range having a lower value of about 4 J/cm2 tissue, more preferably about 6 J/cm2. The range may have an upper value of 12 J/cm2 tissue, more preferably about 10 J/cm2 tissue. Most preferably, the dose may be about 8 J/cm2 tissue.

This treatment protocol may also be suitable for treating eczema, boils, cold sores (on lips, genitalia or inside mouth), shingles, post-herpetic neuralgia, acne, boils, tooth abscesses, cysts (inflamed or seborrhoiec), warts, osteoitis/bone infection, peri-anal abscesses, genitalia infections, vaginitis, Bell's palsy, swimmers/tropical ear, tinnitus, athlete's, foot, fungal infections of the skin and nails, hot spots, hyaloma tick bite wounds, pododermatitis, rodent ulcers, acral lick granuloma, cellulites, infected wounds, and snuffles.

Typically, when treating tissue trauma or muscle pain, the dose may be in a range having a lower value of about 3 J1 cm2 tissue, more preferably about 4 J/cm2. The range may have an upper value of 7 J/cm2 tissue, more preferably about 6 J/cm2 tissue. Most preferably, the dose may be about 5 J/cm2 tissue.

This treatment protocol may also be suitable for treating: neuropathy, burning of hands and feet, carpal tunnel syndrome, peripheral nerve injuries, sciatica, vascular pathology, Burger's disease, Raynaud's disease, varicose veins, vasculitis, and thrombophlebitis, 10 muscle trauma, muscle spasms (e.g. neck and back spasms), fibromyalgia, repetitive/over-use syndromes, muscle strains, muscle contusions, muscle surgery, muscle pain/myalgia, strains, sprains, inflammation, bruising, surgery, ITB/runners knee, fasciitis, plantar fasciitis, tendonitis, synovitis tension headache and migraine associated with muscle spasms, joint injuries, pain associated with dislocations, inflammation, 15 athralgia, tennis and golfers' elbow, shin splints, trigger points, rotator cuff syndromes, acupuncture points, trigger points, fibrositis, arthritic conditions e.g. DJD (degenerative joint disease), rheumatoid arthritis, gout, cartilage damage (e.g. meniscus bruisingltears), disc injuries/degeneration (e.g. slipped disc), synovitis, metatarsal pain and inflammation, calcaneal spur, bunions, hammer-and-claw toe, fractures (assists in faster callus formation), prevention of bone resorption, treatment of stress fractures, tempero-mandibular conditions, fibro-athralgia, spinal column pain associated with pain in the neck and back, chondrotic tearing, post-operative orthopedic surgery, fractures and osteo trauma.

Typically, when treating swelling, edema, or localized inflammatory processes, the dose may be in a range having a lower value of about 1 J/cm2 tissue, more preferably about 2 J/cm2. The range may have an upper value of 5 J/cm2 tissue, more preferably about 4 J/cm2 tissue, Most preferably, the dose may be about 3 J/cm2 tissue.

This treatment protocol may also be suitable for treating: deep hematomas, inflammation, alopecia, effecting skin re-pigmentation, dental trauma, post-operative ear, nose and throat surgery, seromas, skin hematomas, cellulites, general swelling (including post-operative swelling), lymph edema, lymphatic damage, mastitis, inflammation, vasculites, swelling in the ear canal.

When in the pulsed emission mode, the selected photonic emission parameters may be pre-programmed to produce a range of emitted pulse frequencies for each pre-programmed protocol, each frequency in the pulsed emission mode being selected from the group comprising at least two of the following ranges of frequencies: about 1 20-300 Hz, 301-400 HZ, 401-500 HZ, 501-600 HZ, 601-700 HZ, 701-800 HZ, 801-900 HZ, 901-1000 HZ, 1001-1 100 HZ, 1101-1 101-1200 HZ, 1201-1300 HZ, 1301-1400 HZ, 1401-1500 HZ, 1501-1600 HZ, 1601-1700 HZ, 1701-1800 HZ, 1801-1900 HZ, 1901-2000 HZ, 2001-2100 HZ, 2101-2200 HZ, 2201-2300 HZ, 2301-2400 HZ, 2401-2500 HZ, 2501-2600 HZ, 2601-2700 Hz, 2701-2800 Hz, 2801-2900 Hz, 2901-3000 Hz, and the range from about 16,000 to about 22,000 Hz.

For acute wounds, the pulse repetition rate may be divided equally between 120-300 Hz; 500; 900 Hz; and 2000-2400 Hz.

For chronic wounds, the pulse repetition rate may be divided equally between 120-300 Hz; 400-600 Hz; and 700-820 Hz.

For oral or mucosal pathologies, sinusitis, rhinitis, snuffles, or the like, the pulse repetition rate may be divided equally between 120-300 Hz; 400-600 Hz; and 700-900 Hz.

For swelling, seromas, or haemotomas, the pulse repetition rate may be divided equally between 120-300 Hz; 400-600 Hz; 700-900 Hz; and 2300-5000 Hz.

For tissue trauma, joint, bone/osteo, or arthritic-related complications, the pulse repetition rate may be divided equally between 120-300 Hz; 500-800 Hz; 801-1200 Hz; and 2000-4700 Hz.

For abscesses, acne, and other infection-related complications, the pulse repetition rate may be divided equally between 200-500 Hz; 600-800 Hz; 850-1 500 Hz; and 17000-20000 Hz.

The parameters may be selected so that in the event that an unsophisticated user selects or activates an incorrect or inapplicable treatment protocol, the overlapping pulsed frequencies will deliver a treatment protocol that is at least partially effective for, or may provide partial relief from, any of the abovementioned conditions.

When a sufficient dose of light, as pre-programmed into each treatment protocol, has been delivered to a particular area of a wound or lesion, a user of the photonic emission device may be prompted by the device to move the photon emitting source to a further site for treatment. Each movement of the photon emitting source may be prompted by a visual, audio or tactile indicator. This allows a user sufficient time to correctly position the photon emitting source prior to the following treatment cycle being activated.

The audio indicator may be in the form of a beep or buzzer or other easily discernible sound. The visual indicator may be in the form of an interruption in the visible light emission from the probe emitting source, and/or a light flashing on a display panel of the control unit. The light may by interrupted for a period of about 1 to 4 seconds in order to allow users sufficient time to correctly position the probe between treatment cycles. The tactile indicator may be in the form of a vibration emitted by the photon emitting source or a housing in which it may be contained.

The photon therapy device may be powered by conventional batteries, rechargeable batteries, mains power, or solar energy. When powered by solar energy, the device may have a solar panel incorporated therein or associated therewith.

The photon therapy device may include a plurality of activation or protocol buttons displayed on a touch screen made of a water impervious or repellent material, such as a plastics material. The photon therapy device may be made of, a high-impact plastics material to minimize damage to any componentry, the box, the probe, or the photon emitting source.

The invention extends also to a kit, the kit including a controller unit box incorporating a central processing unit as described hereinbefore, a plurality of buttons, each capable of activating a desired pre-programmed treatment protocol, and at least one probe including a photon emitting source, as described hereinbefore. Additionally, the kit may include an instruction booklet or pamphlet, and a battery charger.

Further aspects of the invention will now be explained, by way of example only, with reference to the following drawings.

### DRAWINGS

In the drawings:

FIG. 1 shows a three dimensional drawing of a photon therapy device of the invention;

FIG. 2 shows a schematic drawing of a control panel of a photon therapy device of the invention;

FIG. 3 shows a circuit diagram for one embodiment of a photon therapy device of the invention;

FIG. 4 shows a circuit diagram for another embodiment of a photon therapy device of the invention.

### DETAILED DESCRIPTION OF AN EMBODIMENT OF THE INVENTION

With reference to the drawings, reference numeral 10 is used to indicate generally a photon therapy or light therapy device of the invention.

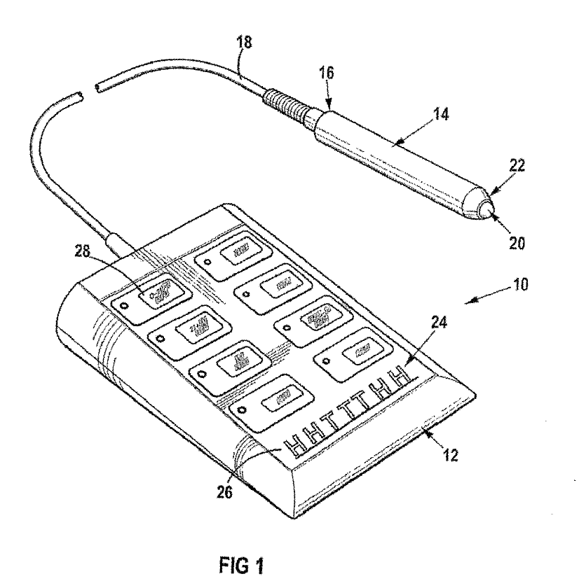

In an embodiment shown in FIG. 1, a photon therapy device 10 includes a generally wedge-shaped controller unit box 12, and a probe 14.

The probe 14 is manufactured from anodized aluminum which is able to withstand reasonable neglect and abuse by users. The probe 14 has a first end 16 to which a cable 18 is attached. The cable 18 is commercially available robotic cable and serves to connect the probe 14 to the controller unit box 12. The cable 14 is easily disconnectable from the probe 14 or box 12, to facilitate easy cleaning of the probe 14 without the possibility of water damage or other damage being inflicted on the controller unit box 12 during the cleaning process.

The probe 14 contains a high-powered 20V LED array unit 20 at an opposed end 22 thereof.

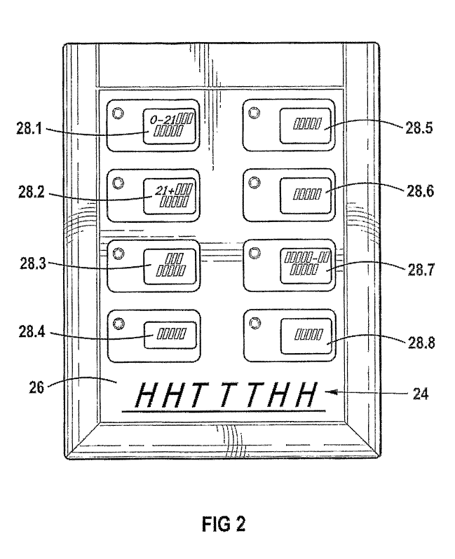

The controller unit box 12 is manufactured from high-impact ABS plastic. To an operatively upwardly facing surface 24 of the box 12 is attached a control panel 26 made of thin section reverse-printed plastic sheeting. The control panel 26 has easily legible and understandable buttons printed thereupon as shown generally by reference numeral 28 (in 5 FIG. 1, and shown in greater detail in FIG. 2), to facilitate easy identification and to ensure easy activation of the desired protocols associated with each button. The controller unit box 12 is wedge-shaped, which facilitates use thereof by patients having impaired vision or mobility, as the control panel 26 is slightly tilted towards the user when in use. In addition, as the control panel 26 is made from a water impervious plastics material, it serves to protect the innards of the controller unit box 12 from accidental water or solvent spillage.

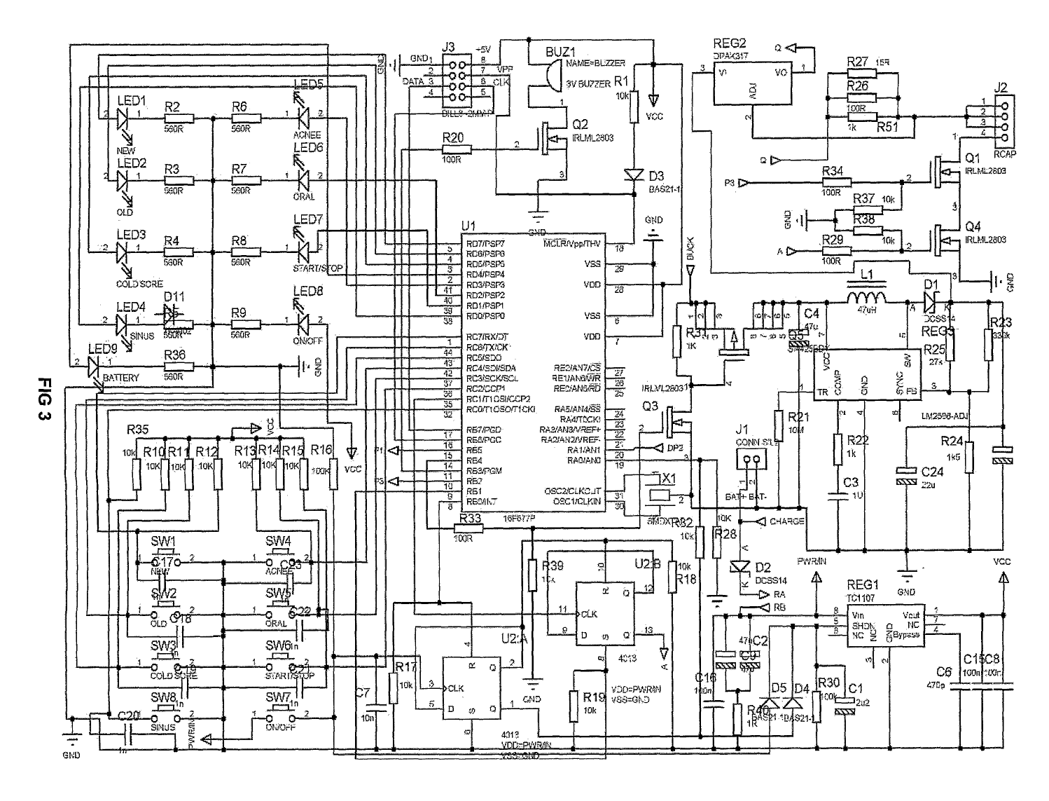

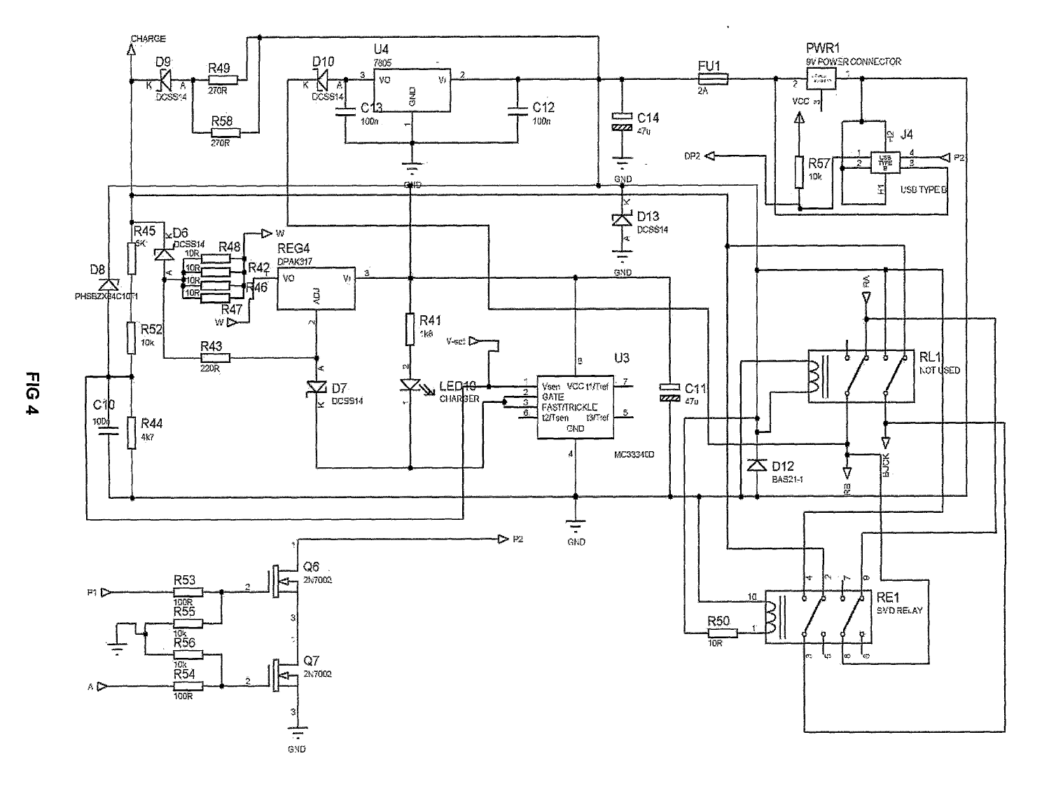

As shown in FIG. 2, various indicia in the form of buttons 28.1-28.8 are printed on the control panel The indicia 28.1-28.8 each activate a tactile button (not shown) proximal to the underside of the control panel 26, activation of each such button by a user serving to activate a desired protocol which has been pre-programmed into a central processing unit (CPU—shown in FIGS. 3 and 4). Each such pre-programmed protocol regulates the photonic emission of the LED 20 located at the first end 22 of the probe 14.

In one embodiment of the invention, the treatment protocol indicia 28.1-28.6 are marked as follows:

0-21 DAY WOUND (28.1);

21+ DAY WOUND (28.2);

ORAL PATHOLOGY (28.3);

SWELLING (28.4);

TISSUE TRAUMA (28.5);

INFECTION (28.6).

Other indicia, which do not include treatment protocols are marked as follows:

START/STOP (28.7); and

ON/OFF (28.8)

It is to be understood that the treatment protocol indicia 28.1-28.6 described above are shown by way of example only and a variety of indicia may be included in various embodiments of the invention, each one specifying a desired treatment protocol for other diseases not included in the abovementioned embodiment.

In addition to the various indicia 28.1-28.8, each button is located adjacent a related LED (30.1-30.8) which indicates whether the related button has been pressed by a user. In addition, certain LEDS such as a BATTERY LOW LED (not shown), are activated by the control unit itself in response to certain pre-programmed parameters being exceeded, as discussed below with reference to FIGS. 3 and 4.

The photonic emission (not shown) from the high-power LED 20 (FIG. 1) for any given protocol has a wavelength of about 640 nm (10%). Optical power of the LED 20 is about 100 mW. When in pulsed wave mode, the photonic emission is about 50 mW. Depending on the desired treatment protocol, the predetermined photonic emission parameters provide a suitable dose per cm2 tissue.

For example, when treating a wound of less than about 21 days old, the pre-programmed dose is about 2 J/cm2 tissue. This treatment protocol is also suitable for treating: Cuts; scratches; scrapes; blisters; surgical wounds; burns; bedsores; superficial bruising; lip wounds; mosquito and other insect bites; contact dermatitis; dermatitis following cancer radiation therapy; and to accelerate healing of skin grafts.

When treating a wound older than about 21 days, the pre-programmed dose is about 4 J/cm2 tissue. This treatment protocol is also suitable for treating: in the tissue: hemorrhoids (protruding); Burger's disease; Raynaud's disease; and neuropathy.

When treating oral pathologies, the dose is about 6 J/cm2 tissue. This treatment protocol is also suitable for treating: sinusitis, tooth abscesses; wounds following tooth extraction; pain following root canal procedures; hypersensitive gums; gingivitis; hypersensitive dentine; post-operative pain following dental implantation; mucositis following cancer radiation therapy; upper respiratory tract infections and swelling; and tonsillitis.

When treating swelling or the like, the pre-programmed dose may be about 3˜1 cm' in the tissue. This treatment protocol is also suitable for treating: inflammation, seromas, skin hematomas, cellulites, general swelling (including post-operative swelling), lymph edema, lymphatic damage, and mastitis.

When treating tissue trauma or muscle pain, the dose is about 5˜1 cm˜ in the tissue. This treatment protocol is also suitable for treating: muscle, osteo, ligament, tendon, and nerve trauma.

When treating infections, abscesses, acne, or viral infections, the pre-programmed dose may be about 8 J/cm2 tissue. This treatment protocol is also suitable for treating: shingles; other related Herpes simplex infections; Herpes zoster infections; post-herpetic neuralgia; Bell's palsy; warts; and chicken pox.

The treatment protocols each include a period of pulsed emission, followed by a period of continuous emission. The treatment protocols include a pulsed photonic emission mode for a time equivalent to about 30% of the total irradiation time, followed by a continuous emission mode for about 70% of the desired treatment protocol. When in continuous mode, the photonic emission is applied at a full (or 100%) duty cycle. Accordingly, when the photonic emission is applied in pulsed mode, the pulse is applied at a duty cycle of 50%.

As an example, when treating a 0-21 day wound and associated conditions as specified hereinbefore, the selected photonic emission parameters include the following discrete frequencies: 146, 147, 266, 292, 294, 528, 587, 727, 802, 880, and 2128 Hz (each frequency 10%), each pulsed for similar lengths of time.

As a further example, when treating a wound older than about 21 days and associated conditions as specified hereinbefore, the selected photonic emission parameters include the following discrete frequencies: 146, 147, 266, 292, 465, 584, 587, 727, 787, 802, 880, 2336, 2349, and 20,000 Hz (each frequency +lo %), each pulsed for similar lengths of time.

When treating infections, abscesses, acne, and associated conditions as specified hereinbefore the selected photonic emission parameters include the following discrete frequencies: 292, 465, 690, 727, 787, 880, 17024, and 20,000 Hz (each frequency +lo %), each pulsed for similar lengths of time.

In addition, when treating oral pathologies and associated conditions as specified hereinbefore, the selected photonic emission parameters include the following discrete frequencies: 146, 444, 465, 522, 727, 760, 776, 787 802, and 880 Hz (each frequency, +lo %), each pulsed for similar lengths of time.

Typically, when treating swelling and associated conditions as specified hereinbefore, the selected photonic emission parameters include the following discrete frequencies: 146, 147, 148, 428, 440, 444, 522, 580, 587, 727, and 787 Hz (each frequency &lo %), each pulsed for similar lengths of time.

Typically, when treating issue trauma and associated conditions as specified hereinbefore, the selected photonic emission parameters include the following discrete frequencies: 146, 294, 587, 660, 727, 787, 802, 880, 1174, 2182, 2349, and 4672 Hz.

Following scanning of each such range of frequencies, the photonic emission enters continuous emission mode for each remainder of the treatment cycle.

It follows that, if 1 J of photonic emission is 20 seconds in duration, in the event of using a 50% pulse ratio, 1 J is thus 40 seconds in duration.

By way of example, for an acute wound (less than 21 days old), the doses are calculated as follows:

Ten pulsed frequencies at the frequencies detailed hereinbefore, followed by continuous emission at 100% duty cycle, to deliver 2˜1 cm˜ cycle. This means that 2 J×20 s equates to 40 s. Accordingly, 30% of 40 s equates to 12 s, which must then be multiplied by 2, as the pulsed mode is run at a 50% duty cycle, equating to 24 seconds for the pulsed mode emission. In addition, the continuous emission is 28 s in duration. Accordingly, the 10 pulsed frequencies need to be spread equally over the allocated time, resulting in 2 s being allocated for each pulsed frequency. Thus, the total time allocated to achieve 2˜1 cm˜ tissue is 24 s pulsed and 28 s in continuous wave mode, providing a total treatment time of 52 seconds 1 cm2 tissue.

The doses for each condition to be treated are calculated similarly. The CPU is a 16F877 5 16F877 microchip with an oscillator frequency of 4 MHz

Operation of an embodiment of the device will now be described by way of example only, with reference specifically to the wiring diagram shown in FIG. 3. References to certain components refer to the like-named components shown in FIG. 3.

### On/Off Button

When a user presses the on/off button, a chip U2A is clocked, causing Q1 pin 1 to go high. This activates regulator 1, and supplies the battery voltage to the divider circuit R35 and R39. Switching power on to regulator REG1 will supply the CPU with 3.3.V and, in so doing, starts the relevant program. In turn Q latches the regulator REG1 in the ON position and lights the ON/OFF LED is switched on. When pressed again, U2A is clocked again causing the Q output to go low. This will cause the REG1 to switch off and turn off the machine.

### Start/Stop Button

Once a specific treatment application button (e.g. 21+ DAY WOUND) has been selected, the button begins operation of that application. The relevant button can only be activated once a specific treatment has been selected. When the STARTISTOP button is pressed, the STARTISTOP LED lights up, and the CPU switches on transistor chip U3Q3, following which power is supplied to the relevant STARTISTOP LED. The program then jumps to the specific treatment loop, which loop controls Q3, Q1 and Q4. Q3, Q1 interrupts power to the high-power treatment LED in the treatment probe (reference numeral 12 in FIG. I).

### Treatment Protocol Buttons

When a treatment button is pressed by a user, the relevant treatment protocol LED is activated and the high power LED in the treatment probe (reference numeral 12 in FIG. 1) is activated by the CPU. If no other button on the control panel is pressed within a 30 second period, the relevant treatment protocol LED will again be switched off in order to save power. Pressing the STARTISTOP button will commence application of the selected treatment. If no buttons are pressed for a continuous period of 5 minutes following activation, the machine will automatically switch off in order to save power.

### LOW POWER Indicator LED:

If the voltage of the 4.8V battery drops below 4V, the LOW POWER LED will light up, indicating that the batteries must be charged. If the battery voltage drops even lower, the LOW POWER LED will flash eight times, following which the device switches off automatically.

To ensure sufficient coverage of the wound or lesion to be treated, a user must move the probe after every treatment cycle over a particular area of skin, i.e. whenever a certain area has received an adequate irradiation dose. To this end, the device emits a beep sound by switching on a buzzer BUZ1 controlled by Q1 Q2 for 100 ms, and also shuts down the photonic emission of the probe for a period of 3 seconds, before resuming the irradiation.

In another embodiment of the invention, shown in FIG. 4, further circuitry is connected to a second port to which another probe can be connected. This allows a user to select 20 either the first probe only, the second probe only, or both probes simultaneously. This is accomplished by pushing the desired treatment button once to activate only the first probe (indicated by continuous LED emission), twice to activate only the second probe, (indicated by a slow-flashing LED) and three times to activate both probes simultaneously. (indicated by a fast-flashing LED). The device will automatically sense the connection of the additional probe and activate the additional software to run the additional probe, which may have a higher output than the first probe.

This is achieved using the embodiment of the invention shown in the circuit diagram of FIG. 4, wherein activating Q1 and Q4 will switch on the first probe, and activating Q6 30 and Q7 will switch on the second probe.

Clinical Case Studies:

Case 1

Female patient 78 years old with 18 month old venous leg ulcer treated with lntra Site gel and the surrounding skin was protected with zinc paste. On Day 1 photon therapy with the photon therapy device of the invention commenced together with the IntraSite gel-covered with a Meloline pad. Photon therapy was done once a day for 5 minutes. On Day 5 the wound was fully closed and progressing well towards full healing. On Day 9 the wound had healed fully and has not re-occurred for 6 months.

Case 2

Over one year old venous leg ulcer in a male patient (an alcoholic with mild venous insufficiency). Wound was treated with L-Mesitran Soft over the previous months with no relief. On Day 1 photon therapy commenced. The L-Mesitran Soft therapy was continued. Photon therapy was used once a day for 5 minutes. Most of the necrotic tissue and fibrin was gone on Day 3. Wound was fully closed and epithelialized by Day 20 and has not re-occurred for 6 months following treatment.

Case 3

68 year old female patient with large wound following after knee prosthesis and numerous unsuccessful skin grafts. The patient had to stay in the hospital during the week and was scheduled for a final skin graft. The home care nurse suggested to the surgeons to treat the patient with the photon therapy device of the invention during the weekend and postpone the surgery. The doctors agreed to awaited preliminary results. The first photon therapy treatment (once a day for 6 minutes) was started on Day 1. The base treatment was L-Mesitran Soft. This wound was over 1 year old at start of treatment and patient had been in hospital during the week for the past year, only going home on weekends. The wound was only treated on weekends and by Day 3 wound had shows a remarkable improvement. The patient was sent back to the hospital after the weekend and there was no photon therapy treatment for the following two weeks. Two weekends and only 6 Photizo treatments later fully closed the wound. Wound has not re-occurred for 6 months following treatment.

Case 4

Four year old canine with large recurring Actinomyces-infected fistulas. Treatment with photon therapy device of the invention for 5 minutes per day was commenced on Day I, after lancing of fistula. Wound was now 4.5 cm in length. Treatment was repeated once 5 daily. By day 20 wound had fully closed, with no recurrence 6 months later.

Case 5

14 Year old tabby cat with severe snuffles for whole of recorded history. Had been treated unsuccessfully with various chronic medications for more than 5 years by same veterinarian. Treatment with photon therapy device of the invention for 4 minutes a day led to remarkable improvement in patient's ability to smell food. By day 3, most symptoms of snuffles had subsided. Snuffles re-occurred once after three weeks. Chronic use of photon therapy device was prescribed and patient has been free of symptoms for more than 6 months.

Case 6

64 year old diabetic patient with severe venous insufficiency. Scheduled for amputation of foot, several months following amputation of toes. Patient commenced photon therapy for 5 to 6 minutes each day on each foot. Within 3 days lividity had returned and surgery was cancelled. Feet appeared healthy with increased blood supply evident.

The parameters have been selected, especially the pulsed and continuous wave mode parameters, so that in the event that an unsophisticated user selecting or activating an incorrect or inapplicable treatment protocol, the overlapping pulsed frequencies will deliver a treatment protocol that is at least partially effective for, or provide partial relief from, any of the abovementioned conditions, without inflicting harm to the user.

The Inventors are of the opinion that they have invented a photon therapy device which enables unsophisticated users to self-treat a variety of conditions, at the push of one or 30 two buttons. This also allows clinics to have a service unit, in which patients can, with little guidance, treat their own conditions, either with or without the assistance of a nurse of clinician. Furthermore, the device is small and light, enabling it to be transported with ease. Advantageously, the device has no moving parts which may be subject to wear and tear. As a further advantage, the device of the invention allows unskilled users to obtain at least partial relief from a variety of conditions, due to the overlapping nature of the frequencies specified in the pre-programmed treatment protocols.

What is claimed:

1. A controller unit for controlling the emission of at least one photon emitting source in communication with the controller unit, the controller unit including: at least one central processing unit pre-programmed with at least one selectable photonic emission protocol, said photonic emission protocol having included therein parameters for regulating photonic emission from the at least one photon emitting source, wherein the at least one pre-programmed emission protocol has a pulsed emission mode and a continuous emission mode, with a pulse rate selected from a range of frequencies of between 120 Hz and 20,000 Hz when in the pulsed emission mode; and selection means for allowing a user to select the at least one pre-programmed photonic emission protocol.

2. The controller unit as claimed in claim 1, wherein the at least one pre-programmed photonic emission protocol includes pulsed photonic emission for a time period equivalent to about 15 to 50% of the selected emission protocol time.

3. The controller unit as claimed in claim 2, wherein the at least one pre-programmed photonic emission protocol includes pulsed photon emission for a time period equivalent to about 20 to 40% of the selected emission protocol time.

4. The controller unit as claimed in claim 3, wherein the at least one pre-programmed photonic emission protocol includes pulsed photon emission for a time period equivalent to about 30% of the selected photon emission protocol time.

5. The controller unit as claimed in any one of claims 1 to 4, wherein the parameters included in the at least one pre-programmed photonic emission protocol include the pulse frequency, dose, intensity, irradiation time, and continuous or pulsed emission mode of the at least one photon emitting source.

6. The controller unit as claimed in any one of claims 1 to 5, wherein the at least one photon emitting source includes at least one light emitting diode (LED).

7. The controller unit as claimed in any one of claims 1 to 6, which includes circuitry for powering a second photon emitting source in synchronisation with the at least one photon emitting source.

8. The controller unit as claimed in claim 1 to 6, wherein the controller unit includes circuitry for powering the second photon emitting source separately from the at least one photon emitting source.

9. The controller unit as claimed in any one of claims 1 to 8, wherein the wavelengths of light produced by the at least one photon emitting source are near-coherent and are between about 500 nm and about 900 nm.

10. The controller unit as claimed in claim 9, wherein the wavelengths of light produced by the at least one photon emitting source are between about 600 and 700 nm.

11. The controller unit as claimed in claim 10, wherein the wavelengths of light produced by the at least one photon emitting source are in the order of 630 to 640 nm.

12. The controller unit as claimed in any one of claims 1 to 11, wherein the power output of the at least one photon emitting source is about 50 to 150 mW in continuous wave mode.

13. The controller unit as claimed in claim 12, wherein the power output of the at least one photon emitting source is about 80 to 120 mW in continuous wave mode.

14. The controller unit as claimed in claim 13, wherein the power output of the at least one photon emitting source is about 100 mW.

15. The controller unit as claimed in any one of claims 1 to 14, wherein the power output of the at least one photon emitting source is about 30 to 70 mW in pulsed mode.

16. The controller unit as claimed in claim 15, wherein the power output of the at least one photon emitting source is about 40 to 60 mW in pulsed mode

17. The controller unit as claimed in claim 16, wherein the power output of the at least one photon emitting source is about 50 mW in pulsed mode.

18. The controller unit as claimed in any one of claims 7 to 17, wherein the second photon emitting source has a wavelength or power output similar to that of the first photon emitting source.

19. The controller unit as claimed in any one of claims 7 to 17, wherein the second photon emitting source has a wavelength or power output differing from that of the first photon emitting source.

20. The controller unit as claimed in any one of claims 1 to 19, wherein, when in the pulsed emission mode, the selected photonic emission parameters are pre-programmed to produce a range of emitted pulse frequencies for each pre-programmed protocol, each frequency in the pulsed emission mode being selected from the group comprising at least two of the following ranges of frequencies: about 120-300 Hz, 301-400 HZ, 401-500 HZ, 501-600 HZ, 601-700 HZ, 701-800 HZ, 801-900 HZ, 901-1000 HZ, 1001-1100 HZ, 1101-1101-1200 HZ, 1201-1300 HZ, 1301-1400 HZ, 1401-1500 HZ, 1501-1600 HZ, 1601-1700 HZ, 1701-1800 HZ, 1801-1900 HZ, 1901-2000 HZ, 2001-2100 HZ, 2101-2200 HZ, 2201-2300 HZ, 2301-2400 HZ, 2401-2500 HZ, 2501-2600 HZ, 2601-2700 HZ, 2701-2800 HZ, 2801-2900 HZ, 2901-3000 Hz, and the range from about 16,000 to about 22,000 Hz.

21. The controller unit as claimed in any one of claims 1 to 20 including, for treatment of acute wounds, a sequential pulse repetition rate of about 120-300 Hz; 500-900 Hz; and 2000-2400 Hz.

22. The controller unit as claimed in any one of claims 1 to 21 including, for treatment of chronic wounds, a sequential pulse repetition rate of about 120-300 Hz; 400-600 Hz; and 700-820 Hz.

23. The controller unit as claimed in any one of claims 1 to 22 including, for treatment of oral or mucosal pathologies, sinusitis, rhinitis, snuffles, or the like, a sequential pulse repetition rate of about 120-300 Hz; 400-600 Hz; and 700-900 Hz.

24. The controller unit as claimed in any one of claims 1 to 23 including, for swelling, seromas, or haemotomas, or the like, a sequential pulse repetition rate of about 120-300 Hz; 400-600 Hz; 700-900 Hz; and 2300-5000 Hz.

25. The controller unit as claimed in any one of claims 1 to 24 including, for tissue trauma, joint, bone/osteo, or arthritic-related complications, a sequential pulse repetition rate of about 120-300 Hz; 500-800 Hz; 801-1200 Hz; and 2000-4700 Hz.

26. The controller unit as claimed in any one of claims 1 to 25 including, for abscesses, acne, and other infection-related complications, a sequential pulse repetition rate of about 200-500 Hz; 600-800 Hz; 850-1 500 Hz; and 17000-20000 Hz.

27. The controller unit as claimed in any one of claims 1 to 26 wherein, when a sufficient dose of light, as pre-programmed into each treatment protocol, has been delivered to a particular area of a wound or lesion, a user of the photonic emission device is prompted by means of a visual, audio or tactile indicator to move the photon emitting source to a further site for treatment.

28. The controller unit as claimed in any one of claims 1 to 27 having power control circuitry for being powered by conventional batteries, rechargeable batteries, mains power, or solar energy.

29. The controller unit as claimed in any one of claims 1 to 28, which includes a plurality of pre-programmed photonic emission protocols, each photonic emission protocol being selectable by way of a separate button associated with each such protocol.

30. A photon therapy device including the controller unit as claimed in any one of claims 1 to 29, the photon therapy device including at least one photon emitting source operably connected thereto.

31. The photon therapy device as claimed in claim 30, including a second photon emitting source operably connected thereto.

32. The photon therapy device as claimed in claim 30 or claim 31, wherein the predetermined photonic emission parameters provide an irradiation dose in a range having a lower value of about 0.5 J1 cm2 tissue and a higher value of about 20 J1 cm2 tissue.

33. The photon therapy device as claimed in claim 32, including an irradiation dose of about 2-8 J1 cm2 tissue.

34. Use of a photon therapy device as claimed in any one of claims 30 to 33 for accelerating healing of a wound, lesion or medical condition in an affected area, the wound, lesion, or medical condition being selected from the group comprising: venous ulcers; mouth ulcers; cuts; abrasions; scratches; blisters; surgical wounds; burns; bedsores; superficial bruising; lip wounds; mosquito and other insect bites; contact dermatitis; dermatitis after cancer radiation treatment; accelerating of healing of skin grafts; haemorrhoids (protruding); Burger's disease; Raynaud's disease; neuropathy; herpes simplex infections; herpes zoster infections; cold sores; shingles; post-herpetic neuralgia; Bell's palsy; warts; chicken pox; eczema; boils; tooth abscesses; wounds following tooth extraction; pain following root canal procedures; hypersensitive gums; gingivitis; hypersensitive dentine; post-operative pain following dental implantation; oral mucositis; upper respiratory tract infections and swelling; tonsillitis; and acne, acne-related lesions or other seborrhoeic skin conditions, seromas, skin hematomas, fine lines and wrinkles, crow's feet, blemishes, sun damaged skin, brown age spots, irregular pigmentation, and skin coarseness, scleroderma, sunburn, liver spots, alopecia, cellulites, general swelling (including post-operative swelling), lymph edema, lymphatic damage, mastitis, post traumatic swelling, inflammation, swelling in the ear canal, neuropathy, burning of hands and feet, carpal tunnel syndrome, peripheral nerve injuries, sciatica, vascular pathology, varicose veins, vasculitis, thrombophlebitis, muscle trauma, muscle spasms (e.g. neck and back spasms), fibromyalgia, repetitivelover-use syndromes, muscle strains, muscle contusions, muscle surgery, and muscle painlmyalgia, ligament, tendon and fascia injuries such as strains, sprains, inflammation, bruising, post-operative orthopedic conditions, ITBIrunner's knee, fasciitis, plantar fasciitis, tendonitis, synovitis, tension headache and migraine resulting from muscle spasms, joint injuries, inflammation, athralgia (painful joints), tennis and golfer's elbow, shin splints, trigger points, rotator cuff syndromes, acupuncture points, trigger points, fibrositis, arthritic conditions e.g. DJD (degenerative joint disease), rheumatoid arthritis, gout, cartilage damage (e.g. meniscus bruisingltears), disc injuriesldegeneration, synovitis, metatarsal pain and inflammation, calcaneal spur, bunions, hammer-and-claw toe, fractures, stress fractures, tempero-mandibular conditions, fibro-athralgia, spinal column pain associated with pain in the neck and back, oral and mucosal pathologies such as skin breaks following dental work, acute and chronic sinusitis, rhinitislrhinopathy, inflammatory skin conditions, gingivitis, hypersensitive dentine, oral mucositis, oral wounds, lip wounds, nose fractures, post-operative ear, nose, throat and oral surgery, mouth ulcers, tooth abscesses, pain following root canal procedures, throat infections, painful mouth nerve conditions, conditions that involve an infective process, e.g. certain bacterial, viral, and fungal infections, cold sores (on lips, genitalia or inside mouth), shingles, post-herpetic neuralgia, tooth abscesses, cysts (inflamed or seborrhoiec), warts, osteoitislbone infection, peri-anal abscesses, genitalia infections, and vaginitis, swimmers/tropical ear, tinnitus, athlete's foot, skin and fungal infections, veterinary applications such as othematoma, hot spots, pododermatitis, stomatitis, hygromas, rodent ulcers, useful in effecting skin re-pigmentation, over use syndromes in equines, acral lick granuloma, bite wounds, cysts, hyaloma tick bite wounds, keratosis, and de-gloving wounds; comprising the steps of selecting a pre-programmed treatment protocol suitable for treating the relevant wound, lesion, or medical condition; and applying the photon emitting source at close range to the affected area and displacing the photon emitting source around and over the affected area until the selected protocol terminates.

35. A kit including: a controller unit as claimed in any one of claims 1 to 29; at least one photon emitting source operably connectable to the controller unit; and instructions for operating the controller box and photon emitting source.

36. The kit as claimed in claim 35 including a second photon emitting source.
